(19) United States
(12) Patent Application Publication
(10) Pub. No.: US 2016/0184572 A1
(43) Pub. Date: Jun. 30, 2016

(54) Disposable Protective Barrier for a Handle of an Ink Application Device and Method of Using the Same Barrier for Tattooing or for Making Permanent Make-Up

(71) Applicant: Long XIAO, Shenzhen, Guangdong (CN)

(72) Inventor: Long XIAO, SHENZHEN, GUANGDONG (CN)

(21) Appl. No.: 14/901,143

(22) PCT Filed: Jul. 3, 2014
(86) PCT No.: PCT/CN2014/081609

§ 371 Date: Dec. 28, 2015

(30) Foreign Application Priority Data
Jul. 6, 2013 (CN) 201320420592.3

(51) Int. Cl. A61M 37/00

(52) U.S. Cl. CPC A61M 37/0076; A61B 2017/0023

(57) ABSTRACT

A disposable protective barrier (10) includes a cover member (11) and an inner separator member (12) extending from the cover member (11). The cover member (11) has an outer wall (110) and an end wall (112) extending from one end of the outer wall (112). A first opening (113) is defined at the other end of the outer wall (112). The inner separator member (12) extends inwards from the end wall (112) of the cover member (11). A handle receiving space (116) is defined between the cover member (11) and the inner separator member (12) for receiving the handle (200) of the ink application device therein. The first opening (113) provides access to the handle (200). The inner separator member (12) has a side wall extending inwards from the end wall (112) of the cover member (11), and an end portion (121) formed at the free end of the side wall (120). A second opening (122) is defined at the other end of the side wall (120). A needle module receiving space (126) is defined in the inner separator member (12) for receiving a needle module (300) of the ink application device. The second opening (122) provides access to the needle module (300). The needle module receiving space (126) is separated from the handle receiving space (116) by the inner separator member (12).

### BACKGROUND OF THE INVENTION

1. Field of the Invention

The present invention relates to an ink application device for tattooing or for making permanent make-up, particularly to a protective barrier for a handle of an ink application device and a method of using the same barrier for tattooing or for making permanent make-up for ensuring sterility when treating customers.

2. The Related Arts

A conventional tattoo device generally comprises a needle drive, a handle, a needle and a needle tip. The user assembles these components together before use. U.S. Pat. No. 6,345,553, and 20060020283 and Chinese patent No. 201595866U disclose a tattoo needle module which composes a tattoo needle and a needle tip pre-assembled as one piece. While tattooing the artist often needs to use several size and configuration of needles. These needles need dimensionally suitable tips. With the tattoo needle module the tattoo artist during use can easily interchange between different tattoo needle modules in various configuration, thereby saving installation time.

The basic shape of a handle for a tattoo needle module is a shaft adapted for being held by hand. One end of the handle is provided with a handle neck for connecting with the needle drive of the tattoo device, and the other end of the handle designed for mounting a tattoo needle module is generally provided with a mounting hole having a mounting member that is correspondingly mounted to a mounting portion at the end of a tattoo needle module. A central hole passes through the handle. A tattoo needle driving component passes through the central hole to be connected with a needle drive that has a motor or electromagnet coils. The tattoo needle driving component passes through the central hole of the handle to drive the tattoo needle in the tattoo needle module to move in reciprocating linear motion. The needle moves from the retracted position into the extended position by the propelling force of the needle driving component. The return movement of the needle from the extended position back into the retracted position is accomplished by the force of a helical spring or an elastically extensible part arranged in the needle module housing. This return force is introduced into the needle through the shaft of the needle.

The tattoo needle module revealed by the Chinese patent No. 201595866U does not comprise a sealing member which prevents ink and bodily fluids from entering the handle of the tattoo device. The tattoo needle module revealed by the U.S. Pat. No. 6,345,553 and 20060020283 comprises a diaphragm which prevents ink and bodily fluids in an infected needle module from entering the handle of the tattoo device. Tattoo artists wear disposable gloves which come into contact with ink and bodily fluids as well as the handle of the tattoo device. To prevent the handle of the tattoo device from getting contaminated, they use a plastic film to wrap the handling surface of the handle or they use a plastic bag with an opening to wrap the handle, the opening of the bag is for attaching the tattoo needle module to the handle mounting hole. The two mentioned wrapping methods do not guarantee a waterproof seal for the handle. Some tattoo artists do not wrap the handle while tattooing, then they autoclave the handle after use, but some ink application devices have the handle and the needle drive integrated and manufactured as one piece. These devices cannot be autoclaved because it will damage electronic components in the devices.

### SUMMARY OF THE INVENTION

An object of the invention is to eliminate the above-mentioned disadvantages and to reliably ensure the sterility of a device for each customer, each tattoo and each permanent make-up in a simple manner.

According to the invention, the object is solved by an ink application device for tattooing or for making permanent make-up, comprising a needle drive, a handle, a disposable protective barrier for the handle and a sterilized disposable needle module in which a needle and a needle tip are integrated.

The disposable protective barrier is a separate part from the sterilized disposable needle module. The disposable protective barrier includes a cover member and an inner separator member extending from the cover member.

The cover member has an outer wall and an end wall extending from one end of the outer wall. A first opening is defined at the other end of the outer wall. The inner separator member extends inwards from the end wall of the cover member. A handle receiving space is defined between the cover member and the inner separator member for receiving the handle of the ink application device therein. The first opening provides access to the handle.

The inner separator member has a side wall extending inwards from the end wall of the cover member, and an end portion formed at the free end of the side wall. A second opening is defined at the other end of the side wall. A needle module receiving space is defined in the inner separator member for receiving a needle module of the ink application device. The second opening provides access to the needle module. The needle module receiving space is separated from the handle receiving space by the inner separator member.

The disposable protective barrier is watertight. The inner separator member covers the mounting hole of the handle and prevents the surface of the handle from being infected by ink and bodily fluids from the infected needle module.

When tattooing, the disposable protective barrier covers the handle. The needle module receiving space is inside the mounting hole of the handle. Thereby the tattoo needle module does not contact the handle directly. In the central hole of the handle, a tattoo needle driving component pushes the tattoo needle via the sealed bottom end of the inner separator member. The sealed bottom end of the inner separator member can be made from flexible thin film materials, or it also can be made of a sealing ring and a transfer shaft sealed by the ring.

The user can interchange between different needle modules during use on one disposable protective barrier for one customer. The disposable protective barrier is removed from the handle after the treatment and is disposed of in the usual manner.

Then, it is necessary for the subsequent treatment to put a new disposable protective barrier cover onto the handle and to mount a new sterilized disposable needle module in the needle module receiving space to make the device ready to operate for the next customer. Since the protective barrier and the disposable needle module are taken from sterile packages directly before use, their sterility is ensured without the need for special hygienic measures by the user

The connection between the protective barrier and the handle can be established and released in a simple manner so that the device can also be simply and safely used even by less experienced users.

The protective barrier is made of thin flexible resin or rubber film, the needle module is mounted to the handle through the thin flexible film. The protective barrier can comprise a mount member for mounting the disposable needle module in the needle module receiving space.

Wherein, the disposable protective barrier for a handle of an ink application device is made of thin flexible synthetic resin or rubber.

Wherein, the handle is the holding part of a tattoo equipment or a permanent makeup equipment.

Wherein, the disposable protective barrier for a handle of an ink application device is adapted for receiving the handle and meanwhile receiving and covering the needle drive.

Wherein, the sealed end of the needle module space can be provided with a connecting structure for coupling a tattoo needle and coupling the tattoo needle driving component. The connecting structure ensures the tattoo needle moves in a reciprocating motion together with the tattoo needle driving component

Wherein, the inner separator member is adapted for being inserted into the mounting hole of the handle

Wherein the clearance between the mounting hole of the handle and the mounting portion of the tattoo needle module is adapted for containing the side wall of the inner separator member.

Wherein a layer of plastic foam can be attached to the outer wall of the cover member, or the outer wall can be made of a waterproof plastic foam. Tattooists will feel more comfortable when holding a handle covered by plastic foam for a long period of time.

Wherein, the handle is connected with the needle drive when in use. The handle can be a separate part from the needle drive. The handle and the needle drive also can be integrated and manufactured as one module.

Wherein the needle drive and the handle are integrated and manufactured as one module.

Wherein the disposable protective barrier is packaged in sterile packaging.

Wherein the side wall of the inner separator member has a mounting member for mounting the needle module.

Wherein the end portion of the inner separator member is made of thin flexible synthetic resin or latex.

Wherein the cover member is made of thin flexible synthetic resin or rubber.

Wherein the end portion of the inner separator member has a member for coupling a needle of the needle module.

Wherein the end portion of the inner separator member has a member for coupling the drive.

Wherein the end portion of the inner separator member has an elastomeric seal ring, and a shaft part surrounded and sealed by the elastomeric sealing ring, one end of the shaft part located in the needle module receiving space connects to a needle of the needle module, the other end of the shaft part located in the handle receiving space side connects to the needle drive, the shaft part is disposable, whereby the shaft part and the cover member is discarded after a single use.

The present invention further provides a method of using a disposable protective barrier for a handle of an ink application device for tattooing or for making permanent make-up, comprising the steps of:

providing a needle drive and a handle connected to the needle drive;
providing a disposable protective barrier comprising a cover member and an inner separator member extending from the cover member; the cover member having an outer wall and an end wall extending from one end of the outer wall, a first opening being defined at the other end of the outer wall, the inner separator member extending inwards from the end wall of the cover member, a handle receiving space being defined between the cover member and the inner separator member for receiving the handle of the ink application device therein, the first opening providing access to the handle; the inner separator member having a side wall extending inwards from the end wall of the cover member, and an end portion formed at the free end of the side wall, a second opening being defined at the other end of the side wall, a needle module receiving space being defined in the inner separator member for receiving a needle module of the ink application device, the second opening providing access to the needle module, the needle module receiving space being separated from the handle receiving space by the inner separator member;
putting the handle in the handle receiving space;
putting the side wall and the end wall of the inner separator member into a mounting hole of the handle,
applying and mounting a needle module to the needle module receiving space of the disposable protective barrier;
applying ink to a surface;
removing the disposable needle module from the needle module receiving space; and
removing the disposable protective barrier from the handle.

Wherein the handle and the needle drive are integrated and manufactured as one piece.

The characteristic and the technical solution of the present invention are best understood from the following detailed description with reference to the accompanying figures, but the figures are only for reference and explaining, not to limit the scope of the invention.

### BRIEF DESCRIPTION OF THE DRAWINGS

The technical solution and the beneficial effects of the present invention are best understood from the following detailed description with reference to the accompanying figures and embodiments.

In the figures:

FIG. 1 is an isometric view of a disposable protective barrier for a handle of an ink application device in accordance with an embodiment of the present invention;

FIG. 2 is a partial section view of FIG. 1;

FIG. 3 is a section view of FIG. 1;

FIG. 4 is a partial section view showing the disposable protective barrier of the present invention in installation and use condition;

FIG. 5 is an exploded isometric view of FIG. 4;

FIG. 6 is a partial section view showing a disposable protective barrier for a handle of an ink application device in accordance with another embodiment of the present invention in installation and use condition;

FIG. 7 is an isometric view showing a disposable protective barrier for a handle of an ink application device in production and use condition;

FIG. 8 is a partial section view showing a disposable protective barrier for a handle of an ink application device in accordance with a further embodiment of the present invention in installation and use condition.

### DETAILED DESCRIPTION OF THE PREFERRED EMBODIMENTS

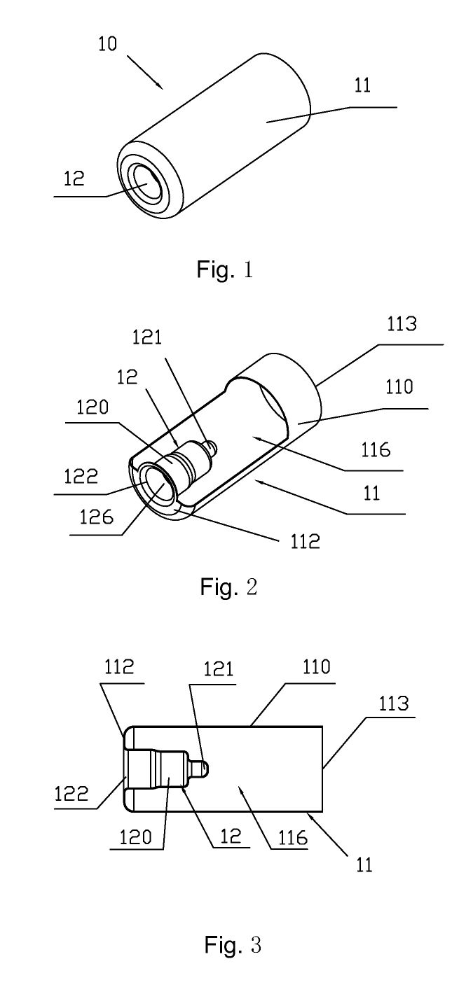

As shown in FIGS. 1-5, a disposable protective barrier 10 for a handle 200 of an ink application device in accordance with an embodiment of the present invention includes a cover member 11 and an inner separator member 12 extending from the cover member 11. The cover member 11 has an outer wall 110 and an end wall 112 extending from one end of the outer wall 110. A first opening 113 is defined at the other end of the outer wall 110. The inner separator member 12 extends inwards from the end wall of the cover member 11. A handle receiving space 116 is defined between the cover member 11 and the inner separator member 12 for receiving the handle 200 of the ink application device therein. The first opening 113 provides access to the handle 200. The inner separator member 12 has a side wall 120 extending inwards from the end wall 112 of the cover member 11, and an end portion 121 formed at the free end of the side wall 120. A second opening 122 is defined at the other end of the side wall 120. A needle module receiving space 126 is defined in the inner separator member 12 for receiving a needle module 300 of the ink application device. The second opening 122 provides access to the needle module 300. The needle module receiving space 126 is separated from the handle receiving space 116 by the inner separator member 12.

The cover member 11 is used to cover the exterior surface of the handle 200, and the inner separator member 12 is used to cover the surface of a mounting hole 210 of the handle 200.

The ink application device can be a tattoo device, which comprises a tattoo machine 400, a tattoo needle driving component 500, a handle 200, a disposable protective barrier 10, and a tattoo needle module 300. The disposable protective barrier 10 is made of thin flexible synthetic resin or latex film. A holding section 220 of the handle 200 is cylindrical with one end being provided with a mounting hole 210 for mounting the tattoo needle module 300. The handle 200 is connected with the tattoo machine 400 via a handle neck 240 disposed at the other end of the handle 200. One end of the tattoo needle driving component 500 is connected with the tattoo machine 400, and the other end is inserted into the central hole 230 defined in the handle 200. The outer wall 110 of the cover member 11 is tubular, of which the shape and size are adapted to cover the holding section 220 of the handle 200, and the size of the first opening 113 is adapted for the handle 200 to be inserted into. The shape and size of the inner separator member 12 are adapted for being inserted into the mounting hole 210 of the handle 200.

The tattoo needle module 300 is provided with a mounting end 310. The size of the mounting hole 210 of the handle 200 is slightly bigger than that of the mounting end 310 of the tattoo needle module 300. The clearance between the mounting hole 210 and the mounting end 310 is adapted for the inner separator member 12 to fit into. When the tattoo needle module 300 is mounted to the handle 200, the mounting end 310 is inserted into the needle module receiving space 126 of the inner separator member 12. In the central hole 230 of the handle 200, the tattoo needle driving component 500 pushes the tattoo needle 320 via the sealed bottom end 121 of the inner separator member 12. After the needle tip of the tattoo needle 320 is pushed out of the needle tip opening 330, an elastic component (not shown) disposed in the tattoo needle module 300 is stretched, and when the tattoo needle driving component 500 returns, the stretched elastic component drags the tattoo needle 320 to the original position, thereby processing a reciprocating motion.

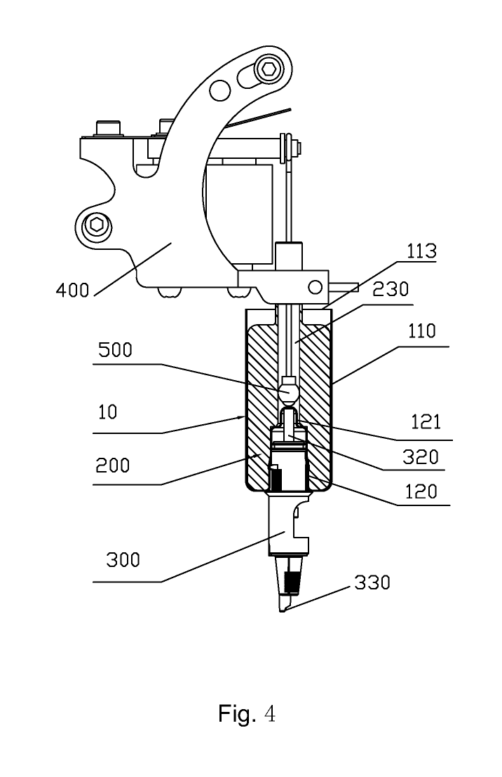

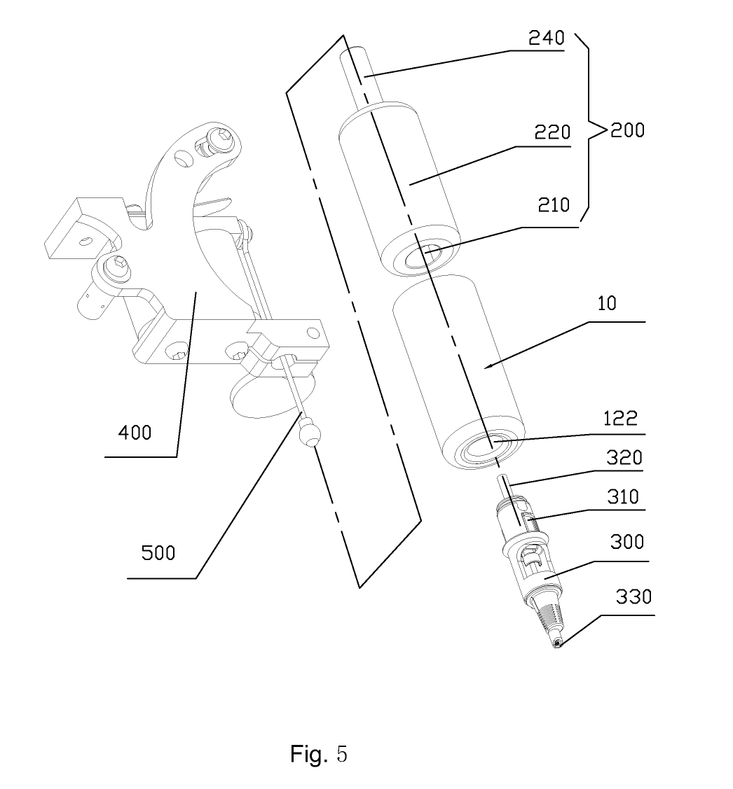

The present invention is further described referring to FIGS. 4-5. The disposable protective barrier 10 of the present invention is packed appropriately and undergoes a process of sterilization. Usually, when a tattooist tattoos the same customer, two to five tattoo needles 320 of different configurations will be used according to the complexity of the tattoo pattern, and these tattoo needles 320 are used alternately. When tattooing the same customer, the tattooist can change between different tattoo needle modules 300 on the same handle 200 and the same disposable protective barrier 10.

While the tattooist is holding a tattoo device in hand, the hands are in contact with the disposable protective barrier 10 without direct contact with the handle 200. The tattoo needle module 300 directly contacts the disposable protective barrier 10 on the handle, and the tattoo needle module 300 does not come into direct contact with the handle 200 and the tattoo needle driving component 500 in the central hole 230 of the handle 200. So, while tattooing, the handle 200 is isolated from the surrounding via the disposable protective barrier 10. After finishing tattooing, first the tattoo needle module 300 is removed, and then the disposable protective barrier 10 is removed from the handle 200. During this process, the handle 200 is isolated from the surrounding.

The size of the first opening 113 of the disposable protective barrier 10 for a handle can be made comparatively big, so the handle 200 can be inserted smoothly for convenience. The size of the cover member 11 of the disposable protective barrier 10 for a handle can be made comparatively big for handles 200 of different shapes and sizes to be inserted smoothly. Meanwhile, the size of the cover member 11 of the disposable protective barrier 10 can be made comparatively big and long, so it is capable of covering both the handle 200 and the body of the tattoo machine 400.

The handle 200 covered by the disposable protective barrier 100 is a separated component from the tattoo machine 400, and while in use, the handle 200 is connected with the tattoo machine 400. In other embodiments, the ink application device for making permanent make-up can integrate the handle and the needle drive together as one module. The handle can also be a holding section of this kind of device. The disposable protective barrier can cover the handle and the needle drive together.

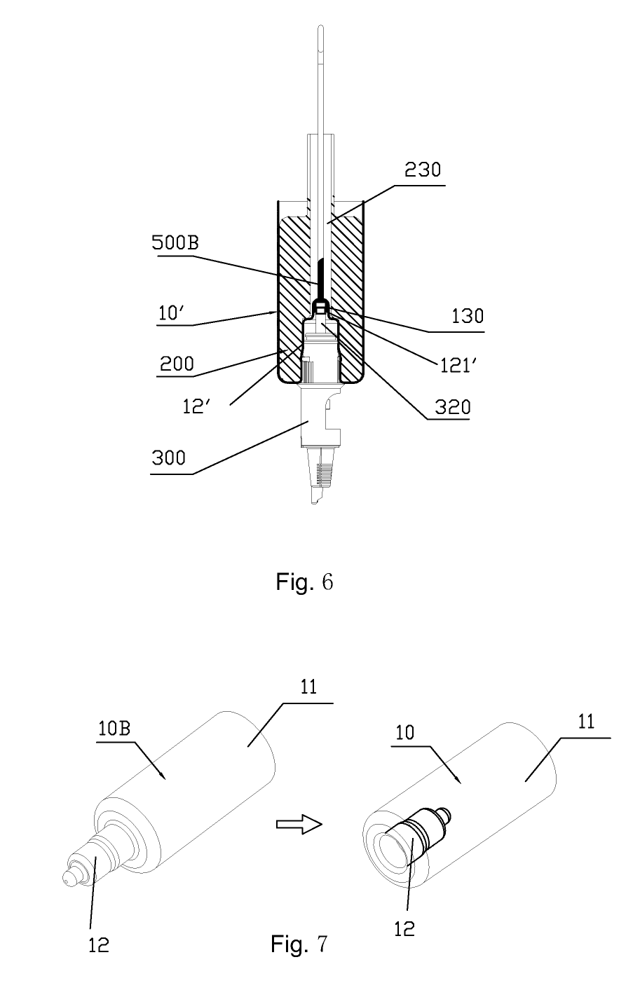

Referring to FIG. 6, as an alternative embodiment, in the disposable protective barrier 10′, an end portion 121′ of the inner separator member 12′ is provided with a connecting structure 130. The connecting structure 130 is coupled with the tattoo needle 320 of the tattoo needle module 300. The end portion 121′ is connected with the tattoo needle driving component 500B in the central hole 230 of the handle 200. In other embodiment, the disposable protective barrier 10′ and the tattoo needle driving component 500B is produced as one body. The advantage of this design is that, when the tattoo needle driving component 500B returns after the tattoo needle 320 is pushed out by the tattoo needle driving component 500B, the tattoo needle 320 can be drawn back by the connecting structure 130, so, the tattoo needle 320 always moves together with the tattoo needle driving component 500B.

Referring to FIG. 7, the shape of the disposable protective barrier 10 can be manufactured in the same shape as the protective barrier 10B. When packing or using the protective barrier, the cover member 11 or the inner separator member 12 can be folded to form the shape of the disposable protective barrier 10. The shape of the protective barrier 10B is easier to manufacture.

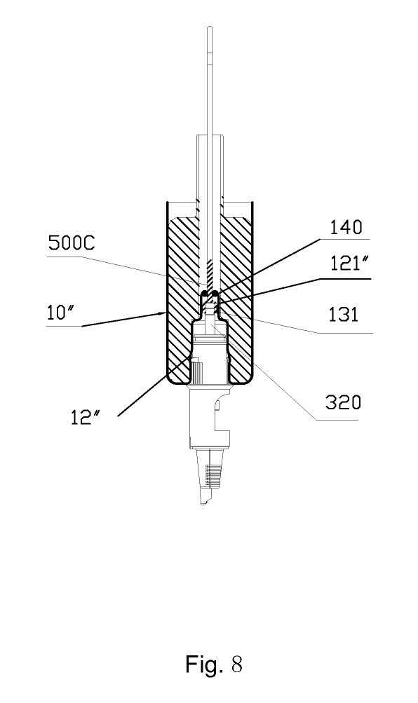

Referring to FIG. 8, in a further embodiment, the end portion 121″ of the inner separator member 12″ of the disposable protective barrier 10″ is provided with a sealing component 140. The sealing component 140 is of an O-ring structure. One end of the tattoo needle driving component 500C passes through the sealing component 140, and connects to the tattoo needle 320 to drive it. A connecting structure 131 is formed at the end of the tattoo needle driving component 500C for directly connecting the tattoo needle 320. The tattoo needle driving component 500C is disposable.

The tattoo needle module can be a preassembled part of a tattoo needle and a needle tip. A tattoo needle and a needle tip of the tattoo needle module can also be separated before use, and when using, the tattoo needle and the needle tip are respectively mounted to a handle and connected with a tattoo needle driving component in the handle.

The present invention further provides a method of using a disposable protective barrier for a handle of an ink application device for tattooing or for making permanent make-up, comprising the steps of:

providing a needle drive and a handle connected to the needle drive;
providing a disposable protective barrier comprising a cover member and an inner separator member extending from the cover member; the cover member having an outer wall and an end wall extending from one end of the outer wall, a first opening being defined at the other end of the outer wall, the inner separator member extending inwards from the end wall of the cover member, a handle receiving space being defined between the cover member and the inner separator member for receiving the handle of the ink application device therein, the first opening providing access to the handle; the inner separator member having a side wall extending inwards from the end wall of the cover member, and an end portion formed at the free end of the side wall, a second opening being defined at the other end of the side wall, a needle module receiving space being defined in the inner separator member for receiving a needle module of the ink application device, the second opening providing access to the needle module, the needle module receiving space being separated from the handle receiving space by the inner separator member;
putting the handle in the handle receiving space;
putting the side wall and the end wall of the inner separator member into a mounting hole of the handle,
applying and mounting a needle module to the needle module receiving space of the disposable protective barrier;
applying ink to a surface;
removing the disposable needle module from the needle module receiving space; and
removing the disposable protective barrier from the handle.

Wherein the handle and the needle drive are integrated and manufactured as one piece.

In summary, the disposable protective barrier for a handle of the present invention covers the handle to isolate the handle from the surrounding during a tattoo operation. While assembling the disposable protective barrier for a handle, a handle can be directly inserted into the disposable protective barrier. The disposable protective barrier can be thrown away after use, thereby avoiding the work of cleaning and high temperature sterilization for tattooists.

It should be understood by those skilled in the art that various modifications and adaptations to the present invention as well as alternative embodiments of the present invention may be contemplated. The preferred embodiments of the present invention disclosed herein are intended to be illustrative only and are not intended to limit the scope of the invention. It is to be understood that the present invention is not limited to the embodiments described above, but encompasses any and all embodiments within the scope of the following claims.

What is claimed is:

1. A disposable protective barrier for a handle of an ink application device comprising:
a cover member and an inner separator member extending from the cover member;
the cover member having an outer wall and an end wall extending from one end of the outer wall, a first opening being defined at the other end of the outer wall, the inner separator member extending inwards from the end wall of the cover member, a handle receiving space being defined between the cover member and the inner separator member for receiving the handle of the ink application device, the first opening providing access to the handle;
the inner separator member having a side wall extending inwards from the end wall of the cover member, and an end portion formed at the free end of the side wall, a second opening being defined at the other end of the side wall, a needle module receiving space being defined in the inner separator member for end portion receiving a needle module of the ink application device, the second opening providing access to the needle module, the needle module receiving space being separated from the handle receiving space by the inner separator member.

2. The disposable protective barrier for a handle of an ink application device of claim 1, wherein the handle connects to a needle drive of the ink application device in use.

3. The disposable protective barrier for a handle of an ink application device of claim 2, wherein the handle receiving space is adapted for receiving the handle and meanwhile receiving the needle drive.

4. The disposable protective barrier for a handle of an ink application device of claim 3, wherein the needle drive and the handle are integrated and manufactured as one module.

5. The disposable protective barrier for a handle of an ink application device of claim 1, wherein the disposable protective barrier is packaged in sterile packaging.

6. The disposable protective barrier for a handle of an ink application device of claim 1, wherein the side wall of the inner separator member has a mounting member for mounting the needle module.

7. The disposable protective barrier for a handle of an ink application device of claim 1, wherein the end portion of the inner separator member is made of thin flexible synthetic resin or latex.

8. The disposable protective barrier for a handle of an ink application device of claim 1, wherein the cover member is made of thin flexible synthetic resin or rubber.

9. The disposable protective barrier for a handle of an ink application device of claim 1, wherein the end portion of the inner separator member has a member for coupling a needle of the needle module.

10. The disposable protective barrier for a handle of an ink application device of claim 2, wherein the end portion of the inner separator member has a member for coupling the needle drive.

11. The disposable protective barrier for a handle of an ink application device of claim 2, wherein the end portion of the inner separator member has an elastomeric seal ring, and a shaft part surrounded and sealed by the elastomeric sealing ring, one end of the shaft part located in the needle module receiving space connects the needle of the needle module, the other end of the shaft part located in the handle receiving space connects to the needle drive, the shaft part is disposable, whereby the shaft part and the cover member is discarded after a single use.

12. A method of using a disposable protective barrier for a handle of an ink application device for tattooing or for making permanent make-up, comprising the steps of:
providing a needle drive and a handle connected to the needle drive;
providing a disposable protective barrier comprising a cover member and an inner separator member extending from the cover member; the cover member having an outer wall and an end wall extending from one end of the outer wall, a first opening being defined at the other end of the outer wall, the inner separator member extending inwards from the end wall of the cover member, a handle receiving space being defined between the cover member and the inner separator member for receiving the handle of the ink application device therein, the first opening providing access to the handle; the inner separator member having a side wall extending inwards from the end wall of the cover member, and an end portion formed at the free end of the side wall, a second opening being defined at the other end of the side wall, a needle module receiving space being defined in the inner separator member for receiving a needle module of the ink application device, the second opening providing access to the needle module, the needle module receiving space being separated from the handle receiving space by the inner separator member;
putting the handle in the handle receiving space;
putting the side wall and the end wall of the inner separator member into the mounting hole of the handle,
applying and mounting a needle module to the needle module receiving space of the disposable protective barrier;
applying ink to a surface;
removing the disposable needle module from the needle module receiving space; and
removing the disposable protective barrier from the handle.

13. The method of using a disposable protective barrier for a handle of an ink application device for tattooing or for making permanent make-up of claim 12, wherein the handle and the needle drive are integrated and manufactured as one piece.
